(19) United States
(12) Patent Application Publication
(10) Pub. No.: US 2004/0006240 A1
(43) Pub. Date: Jan. 8, 2004

(54) Process to prepare 11beta,17alpha ,21-trihydroxy-6alpha-methylpregna-1,4-diene-3,20-dione 21-acetate

(76) Inventor: Bruce Allen Pearlman, Kalamazoo, MI (US)

Correspondence Address: PHARMACIA & UPJOHN, 301 HENRIETTA ST, 0228-32-LAW, KALAMAZOO, MI, 49007, US

(21) Appl. No.: 10/418,485

(22) Filed: Apr. 18, 2003

Related U.S. Application Data
(63) Continuation-in-part of application No. 10/172,267, filed on Jun. 14, 2002, now abandoned.
(60) Provisional application No. 60/299,006, filed on Jun. 18, 2001.

(51) Int. Cl. C07J 5/00

(52) U.S. Cl. 552/570

(57) ABSTRACT

The present invention is a novel process for the transformation of 11β,17α dihydroxy-6α-methylpregna-1,4-diene-3,20-dione 17-acetate (III)

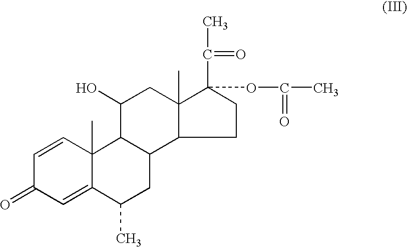

to 11β,17α,21-trihydroxy-6α-methylpregna-1,4-diene-3,20-dione 21-acetate (VI)

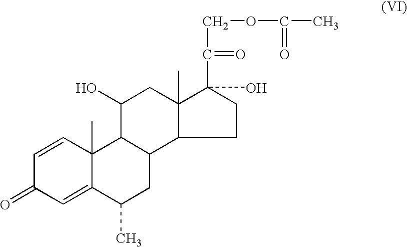

### CROSS-REFERENCE TO RELATED APPLICATIONS

This application is a continuation-in-part under 35 U.S.C. 111(a) of U.S. application Ser. No. 10/172,267, filed Jun. 14, 2002, which claims priority from U.S. Provisional Application No. 60,299,006, filed Jun. 18, 2001.

### BACKGROUND OF THE INVENTION

1. Field of the Invention

The present invention is a process to transform 11β,17α-dihydroxy-6α-methylpregna-1,4-diene-3,20-dione 17-acetate (III) to 11β,17α,21-trihydroxy-6α-methylpregna-1,4-diene-3,20-dione 21-acetate (VI).

2. Description of the Related Art

The functionalization of the C₂₁-methyl group of pregnanes followed by displacement with acetate to produce the corresponding 21-acetate is known to those skilled in the art. GB 2,318,790 discloses the transformation of the C₂₁-methyl group of a Δ¹-11β-hydroxy steroid to the corresponding 21-hydroxy steroid by functionalization with one bromine atom followed by displacement with acetate. The process of the present invention does not use bromine.

GB 2,318,790 discloses the transformation of 17α-hydroxy-6α-methylpregn-4-ene-3,20-dione 17-acetate (I) to 11β,17α-dihydroxy-21-diiodo-6α-methylpregna-1,4-diene-3,20-dione (V) by microbial Δ¹-dehydrogenation by use of Nocardia simplex, microbial 11β-hydroxylation by use of C. lunata and 21-hydroxylation by use of bromine. The present invention transforms 17α-hydroxy-6α-methylpregn-4-ene-3,20-dione 17-acetate (I) to 1β,17α-dihydroxy-21-diiodo-6α-methylpregna-1,4-diene-3,20-dione (V) but does not use bromine.

French patent number 1,243,528 (summarized in CA 55, 24836d) discloses that treatment of 20-oxo steroids with iodine, in the presence of calcium chloride, yields 21,21-diiodo derivatives, which are readily converted into 21-acetoxy steroids by reaction with alkali acetates.

### SUMMARY OF INVENTION

Disclosed is a process for the preparation of 11β,17α,21-trihydroxy-6α-methylpregna-1,4-diene-3,20-dione 21-acetate (VI) which comprises:

(1) hydrolyzing the 11β,17α-dihydroxy-6α-methylpregna-1,4-diene-3,20-dione 17-acetate (III) to produce 11β,17α-dihydroxy-6α-methylpregna-1,4-diene-3,20-dione (IV);

(2) contacting 11β,17α-dihydroxy-6α-methylpregna-1,4-diene-3,20-dione (IV) with iodine, a catalyst, a mild base to produce 1β,17α-dihydroxy-21-diiodo-6α-methylpregna-1,4-diene-3,20-dione (V) and

(3) contacting 1β,17α-dihydroxy-21-diiodo-6α-methylpregna-1,4-diene-3,20-dione (V) with a salt of acetic acid.

Also disclosed is a diiodo steroid of the formula:

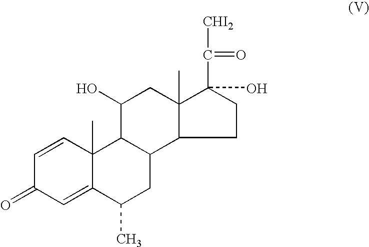

### DETAILED DESCRIPTION OF THE INVENTION

The first two individual steps of the present invention, Δ¹-dehydrogenation (chemical or microbial) and microbial 11β-hydroxylation are known to those skilled in the art. The chemical transformation of a C₂₁-methyl group of a pregnane to the corresponding 21-acetate is also generally known to those skilled in the art. However, the use of the diiodo steroid (V) is novel.

11β,17α-Dihydroxy-6α-methylpregna-1,4-diene-3,20-dione 17 acetate (III) is known and can be produced by known methods from compound (I) as set forth in CHART A.

11β,17α-Dihydroxy-6α-methylpregna-1,4-diene-3,20-dione 17 acetate (III) is deacetylated to give the corresponding 11β,17α-dihydroxy-6α-methylpregna-1,4-diene-3,20-dione (IV). The deacylation or hydrolyzing is accomplished by treatment with a base selected from the group consisting of carbonate, hydroxide or C₁-C₄ alkoxide. It is preferred that the base is selected from the group consisting of carbonate in methanol, hydroxide in aqueous methanol or methoxide. It is more preferred that the base is methoxide. The preferred method is to treat the substrate with sodium methoxide in methanol at about 25°. Ethanol, isopropanol, n-propanol, and other lower alcohols are also operable solvents. Alkoxide salts of other electropositive elements such as potassium, lithium, magnesium, calcium, titanium, aluminum are also operable. The reaction is carried out at temperatures as low as about −40° or as high as about +65°. The preferred temperature range is about 0° to about 25°. The most preferred temperature is about 25° because the reaction is complete in less than 3 hrs. at this temperature.

The 11β,17α-dihydroxy-6α-methylpregna-1,4-diene-3,20-dione (IV) is then 21-acetoxylated to give the desired 11β,17α,21-trihydroxy-6α-methylpregna-1,4-diene-3,20-dione 17-acetate (VI). This 21-acetoxylation is effected by treatment with iodine, a catalyst comprising a metal bromide salt, and a mild base such as calcium hydroxide. It is preferred to use a mixture of calcium oxide, calcium hydroxide, and calcium bromide in methanol. The process is operable with about 1.5-2.5 equivalents of iodine and about 1.0-10 equivalents of calcium hydroxide and/or oxide per equivalents of steroid. The process is operable with as little as 0.05 equivalents of the metal bromide salt. The metal bromide salts, in addition to calcium bromide that may be used in the reaction those that undergo the ion exchange reaction:

metal bromide+calcium hydroxide⇄metal hydroxide+calcium bromide and for which the equilibrium lies to the right. The position of the equilibrium depends on the “hardness” of the metal cation. If the metal cation is a “harder acid” than calcium cation, then it is expected that the equilibrium will lie to the right. For this reaction, the metal bromide salts that are useable are calcium bromide and the bromide salts of metal cations that are harder acids than calcium. Examples of hard acids are listed in Table 1 of the following reference: Pearson, R. G. “Hard and Soft Acids and Bases” J. Am. Chem. Soc. 1963, 85, 3533 and in Table 2.1 of the following reference: Ho, T.-L. “Hard and Soft Acids and Bases Principle in Organic Chemistry” 1977 Academic Press: New York, N.Y.; p. 5. Examples of hard acid metal cations usable in this reaction as the metal cation in the bromide salt are Li+, Na+, K+, Be+2, Mg+, Sr+2, Sn+3, Al+3, Sc+3, La+3, Yb+3, Ga+3, In+3, Cr+3, Fe+3, and Co+.

The present process, which employs bromide salts as catalysts, offers a significant advantage over similar processes in which calcium chloride is used as a catalyst. The Comparative Example shows that when calcium chloride is used as a catalyst, an impurity of a chloro product is produced. This chloro impurity is not desirable, and is difficult to remove.

It is preferred to use 2.0 equivalents of iodine, 1.2 equivalents of calcium oxide, 3.75 eqivalents of calcium hydroxide, and 0.7 equivalents of bromide salt. It is important to add the iodine more slowly than it is consumed to avoid over-iodination which gives rise to 17β-carbomethoxy-6α-methyl-11β,17α-dihydroxyandrosta-1,4-dien-3-one. The reaction temperature should be greater than +10°, preferably greater than +25°, most preferably +25° during the addition of the first half of the iodine in order to avoid formation of 17β-carbomethoxy-6α-methyl-11β,17α-dihydroxyandrosta-1,4-dien-3-one. The reaction temperature should be below +40°, preferably below +25°, most preferably at 0° during the second half of the iodine add in order to minimize degradation of the product diiodide.

The 11β,17α-dihydroxy-21-diiodo-6α-methylpregna-1,4-diene-3,20-dione (V) is finally contacted with a salt of acetic acid, preferably triethylammonium or potassium acetate. However, sodium, magnesium and other metal or amine salt of acetic acid is operable.

### Definitions

The definitions and explanations below are for the terms as used throughout this entire document including both the specification and the claims.

All temperatures are in degrees Celsius.

RPM refers to revolutions per minute.

SCFM refers to standard cubic feet per minute.

TLC refers to thin-layer chromatography.

HPLC refers to high pressure liquid chromatography.

psig refers to pounds per square inch gage.

DO refers to dissolved oxygen.

RO refers to reverse osmosis.

SLM refers to standard liters per minute.

VVM refers to volume per minute.

OUR refers to oxygen uptake rate.

DDQ refers to 2,3-dichloro-5,6-dicyano-1,4-benzoquinone.

Chromatography (column and flash chromatography) refers to purification/separation of compounds expressed as (support, eluent). It is understood that the appropriate fractions are pooled and concentrated to give the desired compound(s).

Pharmaceutically acceptable refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability.

When solvent pairs are used, the ratios of solvents used are volume/volume (v/v).

When the solubility of a solid in a solvent is used the ratio of the solid to the solvent is weight/volume (wt/v).

### EXAMPLES

Without further elaboration, it is believed that one skilled in the art can, using the preceding description, practice the present invention to its fullest extent. The following detailed examples describe how to prepare the various compounds and/or perform the various processes of the invention and are to be construed as merely illustrative, and not limitations of the preceding disclosure in any way whatsoever. Those skilled in the art will promptly recognize appropriate variations from the procedures both as to reactants and as to reaction conditions and techniques.

### Example 1

### Transformation of 11β,17α-dihydroxy-6α-methylpregna-1,4-diene-3,20-dione 17-acetate (III) to 11β,17α-dihydroxy-6α-methylpregna-1,4-diene-3,20-dione (IV)

Sodium methoxide (1.4175 g, 26.2403 mM, 1.05 eq.) in methanol (25%, 6.0 ml) is added to a mixture of 11β,17α-dihydroxy-6α-methylpregna-1,4-diene-3,20-dione 17-acetate (III, 9.9961 g, 24.9578 mM) in methylene chloride (24 ml) and methanol (10 ml) methanol. The mixture is stirred at 20-25° for 2 hours. The reaction is then quenched with acetic acid (1.6 ml, 1.678 g, 27.95 mM, 1.12 eq.), diluted with water/methanol (1/1; 40 ml), stirred at 20-25° for 1 hour, then diluted with water (100 ml) and concentrated under reduced pressure. The residue is diluted with methanol (20 ml) and water (40 ml), concentrated under reduced pressure and the slurry filtered. The cake is washed with water (20 ml) and dried by a nitrogen stream to give the title compound.

### Example 2

### Transformation of 11β,17α-dihydroxy-6α-methylpregna-1,4-diene-3,20-dione (IV) to 11β,17α-dihydroxy-21-diiodo-6α-methylpregna-1,4-diene-3,20-dione (V)

A slurry of 11β,17α-dihydroxy-6α-methylpregna-1,4-diene-3,20-dione (IV, EXAMPLE 1, 30.0050 g, 83.7006 mM), calcium oxide (5.7275 g, 102.13 mM, 1.22 eq.), calcium hydroxide (23.2488 g, 313.79 mM, 3.75 eq.) and calcium bromide (0.5786 g, 2.8946 mM, 0.035 eq.) in methanol (117) at 25° is treated with a mixture of iodine (42.5052 g, 167.47 mM, 2.00 eq.) and calcium bromide (10.897 g, 54.51 mM, 0.65 eq.) in methanol (120 ml) at a steady rate over 4 hours. The reaction mixture is cooled to 0° halfway through the add. The reaction mixture is then poured into a solution of acetic acid (90 ml) in water (2.25 L). The resulting slurry is filtered and the cake is dried by a nitrogen stream to give the title compound.

### Example 3

### Transformation of 11β,17α-dihydroxy-21-diiodo-6α-methylpregna-1,4-diene-3,20-dione (V) To 11β,17α,21-trihydroxy-6α-methylpregna-1,4-diene-3,20-dione 17-acetate (VI)

11β,17α-dihydroxy-21-diiodo-6α-methylpregna-1,4-diene-3,20-dione (V, EXAMPLE 2, 45.0033 g, 73.7433 mM) is added to a mixture of acetic acid (110 ml, 115.4 g, 1.922 moles, 26.1 eq.) and triethylamine (167 ml, 121.2 g, 1.198 moles, 16.2 eq.) in 610 ml acetone. The resulting mixture is stirred at 45° for 2 hours, then cooled to 20-25° and concentrated under reduced pressure. The residue is taken up in methylene chloride (500 ml), washed with aqueous hydrochloric acid (5%, 180 ml) followed by saturated sodium bicarbonate (300 ml) followed by water (340 ml), then filtered through a pad of cartridge grade magnesol (91.72 g), eluting with methylene chloride (1.2 L) followed by acetone/methylene chloride (5/95; 400 ml). The combined eluate is concentrated under reduced pressure to about 400 ml, diluted with methanol (150 ml), and concentrated to about 300 ml. More methanol (150 ml) is added and the mixture is concentrated to about 250 ml. More methanol (100 ml) is added and the mixture is further concentrated, whereupon the product crystallized. The slurry is cooled to −19°, stirred for 2 hours, then filtered. The cake is washed with methanol/water (1/1; 3×20 ml) and dried by a nitrogen stream to give the title compound. A portion of the above solids (3.994 g) is dissolved in methylene chloride/methanol (2/1; 40 ml), concentrated under reduced pressure to about 30 ml, diluted with methanol (10 ml) and concentrated to about 15 ml (2×) to give a slurry which is cooled to −19°, stirred for 2 hours, and filtered. The cake is washed with methanol/water (1/1, 0°; 2×10 ml) and dried by a stream of nitrogen gas. The product had the following composition by LC analysis: 99.19 wt % 6α-methylprednisolone-21-acetate, and 0.14 wt % Δ¹-6α-methyl-11β,17α-dihydroxyprogesterone.

### Comparative Example

### Iodination of Δ¹-6α-Methyl-11β,17α-Dihydroxyprogesterone in the Presence of Calcium Chloride to Form a Mixture of 6α-Methyl-Prednisolone-21-Acetate and 21-Chloro-Δ¹-6α-Methyl-17α-Dihydroxyprogesterone

A mixture of Δ¹-6α-methyl-11β,17α-dihydroxyprogesterone (1.0005 g, 2.7910 mmoles), calcium oxide (0.1915 g, 3.4148 mmoles, 1.22 eq.), calcium hydroxide (0.7754 g, 10.466 mmoles, 3.75 eq.), and calcium chloride (0.0102 g, 0.0919 mmoles, 0.033 eq.) in 4.5 ml methanol (shielded from light) was treated over 2.5 hrs. with a solution of iodine (1.4147 g, 5.5738 mmoles, 2.00 eq.) and calcium chloride (0.203 g, 1.829 mmoles, 0.66 eq.) in 5.3 ml methanol. The addition was carried out using a syringe pump. The temperature was lowered to 0°C. when 90% of the iodine solution had been added. The reaction mixture was then quenched into cold (0° C.) water (75 ml) containing acetic acid (3 ml). The resulting slurry was filtered and the cake washed with water (2×5 ml) and dried by a stream of nitrogen to give 21,21-diiodo-66 ¹-6α-methyl-11β,17α-dihydroxyprogesterone as a crystalline solid. Yield: 1.6093 g (2.637 mmoles, 94.5 chem %).

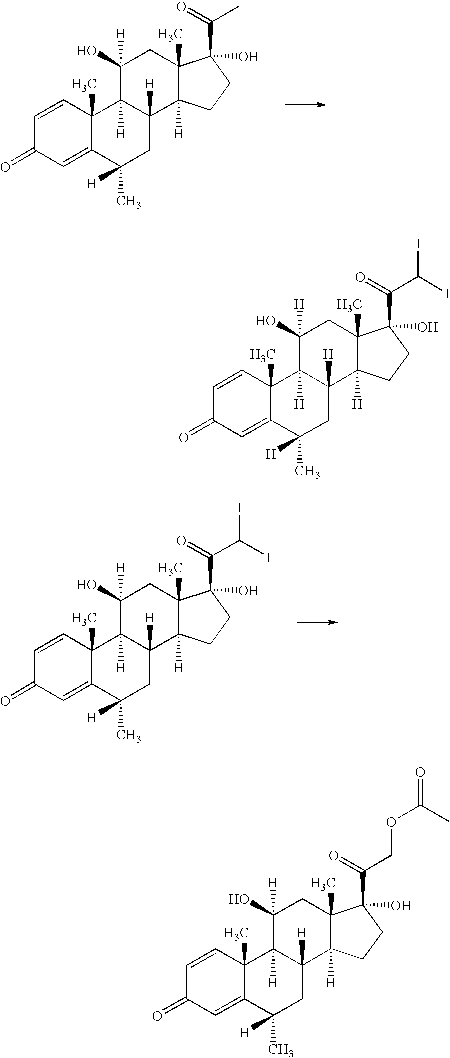

A solution of acetic acid (2.44 ml, 2.560 g, 42.624 mmoles, 26.0 eq.), triethylamine (3.73 ml, 2.708 g, 26.761 mmoles, 16.3 eq.) in 13.5 ml acetone was heated to 55° C. then treated with 21,21-diiodo-Δ¹-6α-methyl-11β,17α-dihydroxyprogesterone (prepared above; 1.0006 g, 1.6396 mmoles). The resulting yellow-orange slurry was stirred at 55° C. for 2 hrs., at which time TLC analysis (eluant: 60/40 ethyl acetate/cyclohexane) indicated that conversion of starting diiodide (R_{f}=0.80) into product 6α-methylprednisolone-21-acetate (R_{f}=0.46) was complete. The reaction mixture was then cooled to +25° C., diluted with 20 ml methylene chloride, washed with 6 ml 0.5 N aq. hydrochloric acid followed by 10 ml sat'd aq. sodium bicarbonate followed by 8.3 ml aq. sodium chloride, then filtered through a pad of magnesol (2.0 g), eluting with methylene chloride (30 ml) followed by 5/95 acetone/methylene chloride (10 ml). The eluate was then concentrated by Rotovap to a volume of 8 ml, then diluted with methanol (˜10 ml) and reconcentrated (2×). The resulting slurry was cooled to −20° C. and filtered. The cake was washed with 0°C. 1/1 methanol/water (2×2 ml) and dried by nitrogen stream to give a crystalline solid. Yield: 0.5058 g (1.2143 mmoles, 74.1%). The product had the following composition by LC analysis: 98.4 wt % 6α-methylprednisolone-21-acetate, 0.15% 6α-methylprednisolone, 0.34 wt % Δ¹-6α-methyl-11β,17α-dihydroxyprogesterone, and 0.30 wt % 21-chloro-Δ¹-6α-methyl-11β,17α-dihydroxyprogesterone.

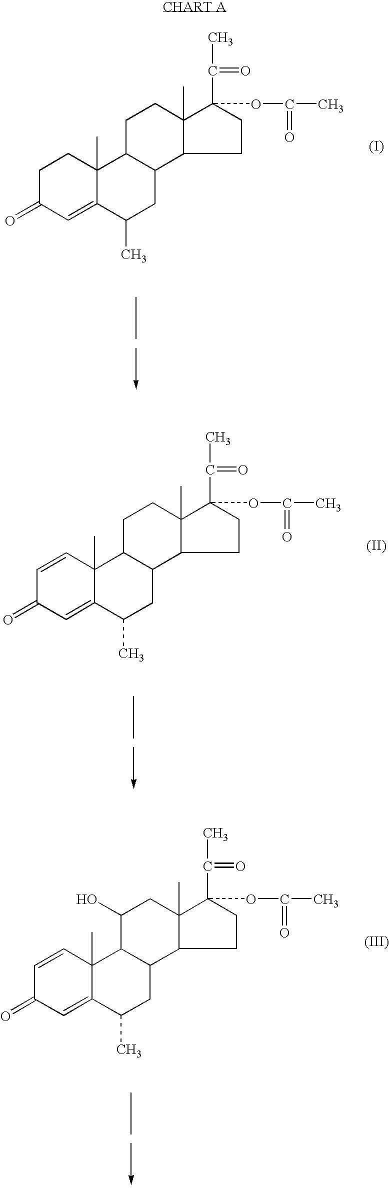

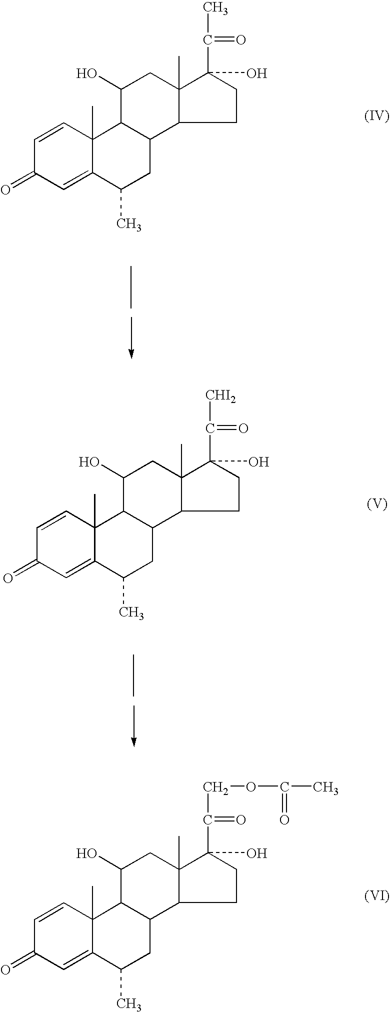

1. A process for the preparation of 11β,17α,21-trihydroxy-6α-methylpregna-1,4-diene-3,20-dione 21-acetate (VI) which comprises:
(1) hydrolyzing the 11β,17α-dihydroxy-6α-methylpregna-1,4-diene-3,20-dione 17-acetate (III) to produce 11β,17α-dihydroxy-6α-methylpregna-1,4-diene-3,20-dione (IV);
(2) contacting 11β,17α-dihydroxy-6α-methylpregna-1,4-diene-3,20-dione (IV) with iodine, a catalyst, a mild base and a metal bromide salt to produce 1β,17α-dihydroxy-21-diiodo-6α-methylpregna-1,4-diene-3,20-dione (V) and
(3) contacting 1β,17α-dihydroxy-21-diiodo-6α-methylpregna-1,4-diene-3,20-dione (V) with a salt of acetic acid.

2. A process for the preparation of 11β,17α,21-trihydroxy-6α-methylpregna-1,4-diene-3,20-dione 21-acetate (VI) according to claim 1 where the hydrolyzing is performed with a base selected from the group consisting of carbonate, hydroxide or C₁-C₄ alkoxide.

3. A process for the preparation of 11β,17α,21-trihydroxy-6α-methylpregna-1,4-diene-3,20-dione 21-acetate (VI) according to claim 2 where the base is selected from the group consisting of carbonate in methanol, hydroxide in aqueous methanol or methoxide.

4. A process for the preparation of 11β,17α,21-trihydroxy-6α-methylpregna-1,4-diene-3,20-dione 21-acetate (VI) according to claim 3 where the base is methoxide.

5. A process for the preparation of 11β,17α,21-trihydroxy-6α-methylpregna-1,4-diene-3,20-dione 21-acetate (VI) according to claim 2 where more than one equivalent of base is used.

6. A process for the preparation of 11β,17α,21-trihydroxy-6α-methylpregna-1,4-diene-3,20-dione 21-acetate (VI) according to claim 1 where the product of step (3) is contacted with iodine in the presence of base and calcium bromide.

7. A process for the preparation of 11β,17α,21-trihydroxy-6α-methylpregna-1,4-diene-3,20-dione 21-acetate (VI) according to claim 1 where the product of step (3) is contacted with iodine in the presence of base and the bromide salt of a hard metal cation.

8. A process for the preparation of 11β,17α,21-trihydroxy-6α-methylpregna-1,4-diene-3,20-dione 21-acetate (VI) according to claim 6 where the base is selected from the group consisting of hydroxide, C₁-C₄ alkoxide.

9. A process for the preparation of 11β,17α,21-trihydroxy-6α-methylpregna-1,4-diene-3,20-dione 21-acetate (VI) according to claim 7 where the base is hydroxide.

10. A process for the preparation of 11β,17α,21-trihydroxy-6α-methylpregna-1,4-diene-3,20-dione 21-acetate (VI) according to claim 6 where the calcium bromide is present at a level of 0.05 to 0.2 equivalents per equivalent of 11β,17α-dihydroxy-6α-methylpregna-1,4-dien-3,20-dione present.

11. A process for the preparation of 11β,17α,21-trihydroxy-6α-methylpregna-1,4-diene-3,20-dione 21-acetate (VI) according to claim 1 where the product of step (4) is contacted with CH₃—COO⁻.

12. A process for the preparation of 11β,17α,21-trihydroxy-6α-methylpregna-1,4-diene-3,20-dione 21-acetate (VI) according to claim 1 where the 11β,17α,21-trihydroxy-6α-methylpregna-1,4-diene-3,20-dione 21-acetate (VI) produced contains not more than 0.1% of any impurity.

13. A diiodo steroid of the formula: [chemical structure]
